(19) United States
(12) Patent Application Publication
(10) Pub. No.: US 2016/0166581 A1
(43) Pub. Date: Jun. 16, 2016

(54) Method for Treating Equine Metabolic Syndrome, Feline Diabetes and Feline Renal Disease

(71) Applicant: Stephen Wills, Washington, WA (US)

(72) Inventor: Stephen Wills, Washington, WA (US)

(21) Appl. No.: 14/572,681

(22) Filed: Dec. 16, 2014

(51) Int. Cl. A61K 31/55; A61K 31/325; A61K 31/473; A61K 31/445

(52) U.S. Cl. CPC A61K 31/55; A61K 31/445; A61K 31/325; A61K 31/473

(57) ABSTRACT

There is disclosed a method for treating Equine Metabolic Syndrome (EMS) comprising administering an acetyl cholinesterase inhibitor compound to the suspected EMS horse. The present disclosure further provides a method for treating feline or canine diabetes and feline or canine renal disease comprising administering an acetyl cholinesterase inhibitor compound to the suspected feline or canine.

### CROSS REFERENCE TO RELATED APPLICATION

This patent application claims priority from U.S. provisional patent application 61/916,631 filed on 16 Dec. 2014

### TECHNICAL FIELD

The present disclosure provides a method for treating Equine Metabolic Syndrome (EMS) comprising administering an acetyl cholinesterase inhibitor compound to the suspected EMS horse. The present disclosure further provides a method for treating feline or canine diabetes and feline renal disease comprising administering an acetyl cholinesterase inhibitor compound to the suspected feline or canine.

### BACKGROUND

Equine Metabolic Syndrome (EMS) is also called Peripheral Cushing's Disease or insulin resistance. EMS is caused when fat cells or adipose tissue produce high levels of adipokines, a protein hormone that leads to an increase in cortisol. As a result of the abnormal hormone production, a horse's normal response to the hormone insulin is disrupted, resulting in high insulin and glucose blood concentrations. The horse cannot properly metabolize carbohydrates, including starches and sugars. This mechanism can trigger the onset of laminitis.

Certain management practices such as feeding high caloric diets to relatively inactive horses can predispose a horse to EMS. Equine Metabolic Syndrome can occur at any point after a horse reaches maturity. Management practices that predispose a horse to EMS are most likely initiated during the first 10 years of a horse's life. Some breeds such as Miniature horses and donkeys, ponies, Paso Finos, and Morgans tend to be more predisposed to this syndrome.

Equine Metabolic Syndrome is usually first recognized when chronic recurrent laminitis is seen in fat horses that lack other laminitis triggers. The most common clinical signs are abnormal intra-abdominal fat deposits (belly fat) as well as fat accumulation in the crest of the neck, over the rump and in the sheath of male horses. These horses will usually have a high body condition score of 7 (Fleshy) or higher. However, other equine may just have abnormal fat deposits. There is currently no single diagnostic test that can definitively diagnose EMS, but elevated levels of glucose and insulin after fasting along with negative results from the dexamethasone suppression test (DST) used to diagnose Equine Cushing's are good indicators of EMS.

It is a disorder of carbohydrate metabolism that is typically seen in overweight middle-aged horses and ponies, and is often associated with severe laminitis. EMS is common in pony breeds, Morgan horses, and Paso Fino horses; however, EMS can affect any breed. Horses with EMS are usually identified as “easy keepers” and historically have not required much feed/energy to maintain weight. A very common physical trait of these horses is the presence of fat deposits in abnormal places. For example, this includes the neck, tail head, and the sides over the ribs. Unfortunately, many of these horses go unnoticed until it is too late and laminitis has already set in.

The exact mechanism is not fully understood, but it has been well documented that high insulin levels in the blood lead to laminitis episodes in the horse. When horses graze on lush pasture, it results in an increase concentration of glucose in the bloodstream. In the normal horse, an increase in glucose will respond by a slight elevation of insulin until the blood glucose levels are normal. However, in the EMS horse, the body doesn't respond like it should to the insulin and much more is released as a result. This sets up a dangerous cascade of events for the horse.

It is recommended to do a simple blood insulin and glucose ratio test on any horses that display the characteristics described. Blood should be drawn following a night of fasting and recommended 12 hours off of grass pasture. This is a useful screening test because high serum insulin concentrations are detected in horses with moderate to severe insulin resistance.

Current treatment is often directed to insulin sensitivity and glucose tolerance by improved by dietary restriction and exercise aimed at reversing obesity. There are some newer commercial feeds that are formulated for a horse with EMS. They are typically lower in starch and sugars, higher in fiber, and supplemented with vitamins and minerals. They may also have additional fat for horses that need more calories in their diets. Lifestyle changes such as increased exercise and improved diet are still the best way to treat this disease.

Medical management of these horses during a laminitic episode is very important. During the acute phase, managing the horse's pain, supporting the hoof, and controlling the insulin levels in the blood are critical to the horse's survival. Pain management is often provided through non-steroidal anti-inflammatory medications. Additionally, the human drug metformin, previously used for type two diabetes, has recently been added to the list of potential helpful medications for insulin resistant horses. Metformin has been shown to increase the sensitivity of insulin but has poor bioavailability (the measure of the amount of drug that is actually absorbed from a given dose) in the horse. Current dosing for metformin is 15 mg/kg twice daily. Therefore, there is a need in the veterinary field for improved treatment options for EMS. The present invention was made to address this need.

Diabetic cats can be treated with oral medications, while others require twice a day insulin injections. Healthy diabetic cats can sometimes be successfully treated with glipizide, an orally administered hypoglycemic medication that lowers blood glucose. Adverse side effects, although uncommon, include vomiting, loss of appetite, and liver damage. If you use glipizide, have your cat's glucose levels checked regularly to verify medication's efficacy. Although glipizide works for some diabetic cats, most require insulin injections to successfully control their disease. In addition, the administration of oral medication on a long-term basis is difficult for many cats and their owners; insulin injections may be a better choice for them. Therefore, there exists a need for better oral feline medications for feline diabetes.

### SUMMARY

The present disclosure provides a method for treating Equine Metabolic Syndrome (EMS) comprising administering an acetyl cholinesterase inhibitor compound to the suspected EMS horse. Preferably, the acetyl cholinesterase inhibitor compound is selected from the group consisting of donepezil, galantamine, rivastigmine, tacrine, combinations thereof, and pharmaceutically acceptable salts thereof. Most preferably, in a horse the daily dosage of donepezil is from about 25 mg to about 150 mg, the weekly dose of donepezil is from about 150 mg to about 600 mg taken two to three times per week. The daily dosage of galantamine is from about 100 mg to about 320 mg, and the daily dosage of rivastigmine is from about 60 mg to about 90 mg.

The present disclosure provides a method for treating feline or canine diabetes, comprising administering an acetyl cholinesterase inhibitor compound to a diabetic cat or dog. Preferably, the acetyl cholinesterase inhibitor compound is selected from the group consisting of donepezil, galantamine, rivastigmine, tacrine, combinations thereof, and pharmaceutically acceptable salts thereof. Most preferably, in a cat the daily dosage of donepezil is from about 1 mg to about 4 mg, the weekly dose of donepezil is from about 10 mg to about 50 mg taken two to three times per week, and wherein the canine dosage is proportional depending on the size of the dog. The daily dosage of galantamine is from about 1 mg to about 4 mg, and the daily dosage of rivastigmine is from about 1 mg to about 3 mg, and wherein the canine dosage is proportional depending on the size of the dog.

The present disclosure further provides a method for treating equine laminitis and prevention of equine coffin bone rotation and descent, such as due to exposure to black walnut, comprising administering an acetyl cholinesterase inhibitor compound to the suspected horse. Preferably, the acetyl cholinesterase inhibitor compound is selected from the group consisting of donepezil, galantamine, rivastigmine, tacrine, combinations thereof, and pharmaceutically acceptable salts thereof. Most preferably, in a horse the daily dosage of donepezil is from about 25 mg to about 150 mg, the weekly dose of donepezil is from about 150 mg to about 600 mg taken two to three times per week. The daily dosage of galantamine is from about 100 mg to about 320 mg, and the daily dosage of rivastigmine is from about 60 mg to about 90 mg.

### DETAILED DESCRIPTION

Acetyl cholinesterase inhibitors increase the amount of neurotransmitter acetylcholine at the nerve terminal by decreasing its breakdown by the enzyme cholinesterase. European Patent 0296560 discloses a number of compounds indicated as acetyl cholinesterase inhibitors useful in the treatment of Alzheimer's disease. (1-benzyl-4->(5,6-dimethoxy-1-indanon)-2-yl methylpiperidine, is also known as donepezil, E-2020 and Aricept®. Suitable doses of the acetyl cholinesterase inhibitor compounds are indicated to be in the range 0.1 to 300 mg, preferably 1 to 100 mg, per adult per day. Examples of active agents for amyloid-related disorders are doxorubicin, galantamine, tacrine (Cognex), metrifonate, rivastigmine, selegiline, physostigmine, donepezil (Aricept), milameline, xanomeline, saeluzole, acetyl-L-carnitine, idebenone, ENA-713, memric, quetiapine, neurestrol and neuromidal. Preferably, the acetyl cholinesterase inhibitor or butylcholinesterase inhibitor is selected from donepezil (Aricept), tacrine (Cognex) rivastigmine (Exelon), physostigmine (Synapton), galanthamine (Reminyl), metrifonate (Promem), quilostigmine, tolserine, thiatolserine, cymserine, thiacymserine, neostigmine, eseroline, zifrosilone, mestinon, huperzine A, phenserine, and icopezil or a pharmaceutically acceptable salt of one of the foregoing compounds.

Acetyl cholinesterase inhibitors are typically administered as a pharmaceutical composition that comprises the acetyl cholinesterase inhibitor that is greater than 95% and preferably greater than 99% pure by weight and one or more excipients, diluents or other inert ingredients commonly found in pharmaceutical compositions.

### Example 1

This example provides the results of an Equine Metabolic Syndrome (EMS)—Insulin Tolerance Test. Four Geldings with body condition score >6 out of 9, consistent with physical findings of EMS are used in an experimental design testing donepezil as an effective agent to improve insulin sensitivity and lower fasting blood glucose. Four horses with physical characteristics of EMS are tested for insulin tolerance. Fasting blood glucose levels are obtained on all 4 horses with the following results:

| | |
|---|---|
| #1- | 132 mg/dl |
| #2- | 151 mg/dl |
| #3- | 144 mg/dl |
| #4- | 167 mg/dl |

All 4 horses are given 0.1 IU/kg of recombinant insulin. Blood sampling is obtained 30 minutes later. The following results are obtained:

| | | |
|---|---|---|
| #1- | 113 mg/dl | (decrease 14%) |
| #2- | 132 mg/dl | (decrease 13%) |
| #3- | 119 mg/dl | (decrease 17%) |
| #4- | 122 mg/dl | (decrease 27%) |

Then, all 4 horses are treated for 4 weeks with Donepezil 75 mg daily. No changes are observed in behavior of animals or feeding habits. Follow up fasting blood glucose levels are obtained:

| | | |
|---|---|---|
| #1 | 108 mg/dl | (18%) |
| #2 | 112 mg/dl | (26%) |
| #3 | 104 mg/dl | (28%) |
| #4 | 121 mg/dl | (27%) |

The same 4 horses are administered 0.1 IU/kg IV recombinant insulin. The following results are obtained:

| | | |
|---|---|---|
| #1 | 56 mg/dl | (decrease 48%) |
| #2 | 61 mg/dl | (decrease 55%) |
| #3 | 57 mg/dl | (decrease 47%) |
| #4 | 52 mg/dl | (decrease 57%) |

No hypoglycemic symptoms are observed (i.e. sweating, agitation). Improvement in insulin sensitivity is attributed to donepezil therapy over 4 week period. Insulin sensitivity is significantly improved after 4 week treatment with donepezil. Insulin sensitivity is increased on average 52%. Fasting blood glucose levels are decreased on average 25%, after treatment with donepezil. Therefore, this study in horses shows the benefits of donepezil treatment for EMS.

### Example 2

This example shows that Black Walnut extract induces laminitis. Three domestic horses, average age 26, are corralled in close proximity to a Black Walnut tree with exposed roots and debris from black walnuts shells, including leaves. The weight of the horses is 1100-1200 lbs. Horses are quartered in a 24×36 square foot area with a Black Walnut tree within the confined area. Over the course of 24 hours all 3 horses develop clinical signs and symptoms of laminitis including; 1. Trembling, 2. Sweating, 3. Bounding digital pulse and 4. Founder's stance.

The earliest horse to develop signs/symptoms occurs in 10 hours and the last horse occurs in 14 hours. The horses are evaluated continuously after 6 hours of exposure to remove them from exposure to the black walnut extract once symptoms occur. After removal from the corralled black walnut tree areas they are given 100 mg of donepezil orally and allowed to move about as tolerated in a separate corral absent repeat exposure to black walnut extract.

The horses are given a second dose of donepezil 100 mg on Day #2 post developing laminitis in forefoot. Subsequent doses are 50 mg on Day #3-7. Horses are given Timothy hay and water, but no other medicinal or mechanical interventions.

Results: Warmth, posturing and trembling resolved by Day #3. Normal activity is noted as early as Day #5, but all subjects appear normal by Day #7. No systemic symptoms are noted after day #7.

All 3 horses recover from the black walnut extract induced laminitis and are pastured for a period of two weeks prior to any repeat exposure. The same 3 horses are used in similar exposure design, as in the original experiment to induce laminitis. The horses are corralled as before and similar induction of forefoot laminitis is observed in roughly the same time intervals. Treatment is begun with 100 mg donepezil orally when clinical symptoms are observed and again they are removed from the black walnut extract environment. Dosing schedule is consistent as before with 100 mg donepezil administered on Day #1, and #2. The dose is tapered to 50 mg orally on Days #3 through #7. Resolution of symptoms and normal movements are observed at Day #5 and completed by Day #7.

No observable lasting lameness or constitutional symptoms are observed. Horse movements demonstrate no abnormalities in terms of gait.

### Example 3

This example shows that donepezil can be used to treat Feline Diabetes. Five obese domestic cats with diabetes (average weight 14.2 lbs.) are on glargine insulin for glycemic control. These feline subjects are treated with 1.25 mg donepezil orally daily with food. All cats are being treated with glargine insulin in bid dosing schedule. The morning blood glucose level prior to treatment with donepezil is recorded. Over a 14 day period morning blood glucose levels (prior to feeding) are recorded.

#1 Cat: 14 lbs. Glargine insulin 2 units bid for blood glucose control. Morning blood glucose level prior to feeding and day before treatment with donepezil feeding is 369 mg/dl. Blood glucose levels in morning are recorded during treatment with 1.25 mg donepezil daily for two weeks. Morning blood glucose levels (prior to feeding) during that 14 day period range from 132 to 178 mg/dl. No untoward side effects on the cat regarding behavior or feeding is noted.

#2 Cat: Weight 13.5 lbs. Glucose control with 3 u. bid. Morning blood glucose prior to feeding and before treatment with donepezil is 249 mg/dl. Treatment with donepezil 1.25 mg daily for two weeks. Morning blood glucose levels are recorded between 88-113 mg/dl. Dose of glargine insulin decrease to 2 units secondary to low blood glucose levels in the morning. No ill effects are incurred in the feline subject when blood glucose levels fell below 100 mg/dl.

#3 Cat: Weight 15.25 lbs. Glucose control with glargine insulin 3 units bid (q12 hr). Morning blood glucose level (prior to feeding) before treatment is 361 mg/dl. During treatment with 1.25 mg of donepezil morning blood glucose levels range from 105-128 mg/dl. There are no negative effects as regards activity, feeding or nocturnal patterns.

#4 Cat: Weight 13 lbs. Glucose control with glargine insulin 1.5 units q12 hr. Morning blood glucose level (prior to feeding) before treatment with donepezil is 278 mg/dl. Treatment with 1.25 mg donepezil daily is begun over a 14 day period. Morning blood glucose levels (prior to feeding) range from 96-117 mg/dl. The sleeping or behavioral pattern is effectively unchanged during this period of time.

#5 Cat: Weight 15 lbs. Glucose control with glargine insulin 3 units bid. Morning blood glucose level (prior to feeding) before donepezil treatment is 305 mg/dl. During 14 day treatment with donepezil 1.25 mg daily, morning blood glucose levels range from 108-126 mg/dl. Nocturnal behaviors are unchanged and the cat experienced normal feeding and behaviors.

Glucose Tolerance Test/Felines. Four feline diabetic subjects (male, age 5-7 years) with newly diagnosed DM (diabetes). Fasting blood glucose levels >200 mg/dl (range 214-255 mg/dl). These 4 cats are subjected to intraperitoneal injection of 2 grams/kg glucose. (ipGTT). Blood glucose levels are obtained via Cephalic iv. catheter at 30 and 60 minutes. The results show:

| time | 0 | 30 | 60 minutes |
|---|---|---|---|
| #1 | | 380 mg/dl | 261 mg/dl |
| #2 | | 374 mg/dl | 270 mg/dl |
| #3 | | 369 mg/dl | 248 mg/dl |
| #4 | | 388 mg/dl | 267 mg/dl |

Each feline subject is treated with donepezil 1.25 mg daily for 4 weeks and subject to ipGTT with 2 gm/kg glucose. The following results are obtained at 30 and 60 minutes following injection:

| Time | 0 | 30 | 60 minutes |
|---|---|---|---|
| #1 | | 269 mg/dl (29%) | 152 mg/dl (42%) |
| #2 | | 292 mg/dl (22%) | 164 mg/dl (39%) |
| #3 | | 278 mg/dl (25%) | 145 mg/dl (41%) |
| #4 | | 304 mg/dl (22%) | 147 mg/dl (46%) |

Accordingly, administration of donepezil for 4 weeks prior to ipGTT testing improved glucose tolerance in treated subjects as compared to values obtained in same subjects prior to treatment with donepezil. The 30 minutes blood glucose levels are on average approximately 24% less than the same cats before treatment with donepezil. 60 minute GTT for donepezil treated cats is on average 42% less than those same cats without donepezil treatment.

### Example 4

This example measures feline renal disease. Six cats with stage 3 and 4 renal disease are treated with donepezil 2.5 mg daily for 4 weeks. The average weight of the domestic cats was approximately 6 lbs. Serum creatinine levels are obtained pre and post treatment with donepezil

1. 5 year old cat with Stage 3 renal disease.
Pre—3.3 creatinine Post 1.8 (decrease 45%)
2. 7 year old cat, Stage 3 renal disease
Pre—4.1 creatinine Post—2.0 (decrease 60%)
3. 7 year old cat, Stage 4 renal disease
Pre—5.4 creatinine Post—1.9 (decrease 65%)
4. 9 year old cat, Stage 4 kidney disease
Pre—6.6 creatinine Post—2.5 (decrease 62%)
5. 6 year old cat, with Stage 3 kidney disease
Pre—4.5 creatinine Post—2.3 (decrease 49%)
6. 10 year old cat, Stage 4 kidney disease
Pre—5.5 creatinine Post—2.6 (decrease 53%)

Accordingly, treatment with donepezil 2.5 mg daily over 4 week period improve creatinine approximately 56% in six cats.

I claim:

1. A method for treating Equine Metabolic Syndrome (EMS) comprising administering an acetyl cholinesterase inhibitor compound to the suspected EMS horse. Preferably, the acetyl cholinesterase inhibitor compound is selected from the group consisting of donepezil, galantamine, rivastigmine, tacrine, combinations thereof, and pharmaceutically acceptable salts thereof.

2. The method for treating Equine Metabolic Syndrome (EMS) of claim 1, wherein the acetyl cholinesterase inhibitor compound is selected from the group consisting of donepezil, galantamine, rivastigmine, tacrine, combinations thereof, and pharmaceutically acceptable salts thereof.

3. The method for treating Equine Metabolic Syndrome (EMS) of claim 2, wherein in a horse a daily dosage of donepezil is from about 25 mg to about 150 mg, and wherein a weekly dose of donepezil is from about 150 mg to about 600 mg taken two to three times per week.

4. The method for treating Equine Metabolic Syndrome (EMS) of claim 2, wherein in a horse the daily dosage of galantamine is from about 100 mg to about 320 mg, and a daily dosage of rivastigmine is from about 60 mg to about 90 mg.

5. A pharmaceutical formulation for daily administration comprising an acetyl cholinesterase inhibitor, wherein in a horse, a daily dosage of donepezil is from about 25 mg to about 100 mg, the weekly dose of donepezil is from about 150 mg to about 600 mg taken two to three times per week.

6. The pharmaceutical formulation for daily administration of claim 5 wherein the acetyl cholinesterase inhibitor compound is selected from the group consisting of donepezil, galantamine, rivastigmine, tacrine, combinations thereof, and pharmaceutically acceptable salts thereof.

7. A method for treating feline or canine diabetes, comprising administering an acetyl cholinesterase inhibitor compound to a diabetic cat or dog.

8. The method for treating feline or canine diabetes of claim 7, wherein the acetyl cholinesterase inhibitor compound is selected from the group consisting of donepezil, galantamine, rivastigmine, tacrine, combinations thereof, and pharmaceutically acceptable salts thereof.

9. The method for treating feline or canine diabetes of claim 8, wherein in a cat a daily dosage of donepezil is from about 1 mg to about 4 mg, a weekly dose of donepezil is from about 10 mg to about 50 mg taken two to three times per week, and wherein a canine dosage is proportional depending on the size of the dog.

10. The method for treating feline or canine diabetes of claim 8, wherein a daily dosage of galantamine is from about 1 mg to about 4 mg, and a daily dosage of rivastigmine is from about 1 mg to about 3 mg, and wherein a canine dosage is proportional depending on the size of the dog.

11. A method for treating equine laminitis and prevention of equine coffin bone rotation and descent comprising administering an acetyl cholinesterase inhibitor compound to the suspected horse.

12. The method for treating equine laminitis and prevention of equine coffin bone rotation and descent of claim 11, wherein the acetyl cholinesterase inhibitor compound is selected from the group consisting of donepezil, galantamine, rivastigmine, tacrine, combinations thereof, and pharmaceutically acceptable salts thereof.

13. The method for treating equine laminitis and prevention of equine coffin bone rotation and descent of claim 12, wherein in a horse a daily dosage of donepezil is from about 25 mg to about 150 mg, a weekly dose of donepezil is from about 150 mg to about 600 mg taken two to three times per week.

14. The method for treating equine laminitis and prevention of equine coffin bone rotation and descent of claim 12, wherein a daily dosage of galantamine is from about 100 mg to about 320 mg, and a daily dosage of rivastigmine is from about 60 mg to about 90 mg.
